(19) United States
(12) Patent Application Publication
(10) Pub. No.: US 2009/0192402 A1
(43) Pub. Date: Jul. 30, 2009

(54) SYSTEM AND METHOD PROVIDING BIOFEEDBACK FOR ANXIETY AND STRESS REDUCTION

(75) Inventor: Stephen B. CORN, Sharon, MA (US)

Correspondence Address: LAHIVE & COCKFIELD, LLP;FLOOR 30, SUITE 3000, ONE POST OFFICE SQUARE, BOSTON, MA, 02109, US

(73) Assignee: ENGINEERED VIGILANCE, LLC, Sharon, MA (US)

(21) Appl. No.: 12/363,467

(22) Filed: Jan. 30, 2009

Related U.S. Application Data
(60) Provisional application No. 61/062,849, filed on Jan. 30, 2008.

(51) Int. Cl. A61B 5/08; A61M 21/00

(52) U.S. Cl. 600/534; 600/27

(57) ABSTRACT

An apparatus, system and method for non-contact monitoring of respiratory and/or cardiac functions that is used to provide appropriate biofeedback to a monitored subject in order to reduce stress and anxiety is discussed. Respiratory and/or cardiac waveforms are generated based on monitored physiologic functions. The generated waveforms are analyzed and compared to a target waveform for the monitored subject. Appropriate biofeedback is generated for the monitored subject based on the analysis of the generated waveform.

### RELATED APPLICATION

This application claims the benefit of a U.S. provisional application entitled “Biofeedback and Anxiety/Stress Reduction Method and Device”, U.S. Provisional Patent Application No. 61/062,849, filed Jan. 30, 2008.

### BACKGROUND

Stress and anxiety affect millions of individuals on a daily basis. Chronic stress and anxiety produce adverse physiologic events that significantly impact morbidity and mortality. Personal and family medical history and occupational requirements can exacerbate stress and anxiety levels. Left untreated, stress and anxiety can greatly diminish quality of life for afflicted individuals.

### BRIEF SUMMARY

Relaxation techniques, biofeedback and self-focus on physiologic processes can positively impact general well-being, mitigate the effects of chronic stress and anxiety and decrease morbidity and mortality. The embodiments of the present invention provide a mechanism for non-contact monitoring of physiologic (e.g.: respiratory and/or cardiac) functions that is used to provide appropriate biofeedback to a monitored subject in order to reduce stress and anxiety. Respiratory and/or cardiac waveforms are generated based on the monitored physiologic functions. The generated waveforms are analyzed and compared to a target waveform for the monitored subject. The target waveform may be determined based on a variety of factors including age, occupation and personal and family medical history. Appropriate biofeedback is generated for the monitored subject based on the analysis of the generated waveform. The biofeedback may take a variety of forms including numerical data and waveform displays and may be accompanied by audible and aromatic feedback designed so that the user consciously or unconsciously can control respiration to approach or maintain the target waveform.

In one embodiment, a biofeedback system for reducing anxiety and stress includes a respiratory waveform detection module, an analysis module and a biofeedback module. The respiratory waveform detection module performs non-contact monitoring of a subject to detect respiratory motion and generates a waveform based on the detected respiratory motion. The analysis module programmatically analyzes the generated waveform based upon pre-determined criteria identifying a target waveform for the subject. The biofeedback module provides biofeedback to the subject to assist the subject in obtaining or maintaining the target waveform. The biofeedback is based on the result of the analysis of the generated waveform.

In another embodiment, an integrated biofeedback apparatus for reducing anxiety and stress includes a respiratory waveform detection module, an analysis module, a biofeedback module and a display surface. The respiratory waveform detection module performs non-contact monitoring of a subject to detect respiratory motion and generates a waveform based on the detected respiratory motion. The analysis module programmatically analyzes the generated waveform based upon pre-determined criteria identifying a target waveform for the subject. The biofeedback module provides biofeedback to the subject to assist the subject in obtaining or maintaining the target waveform. The biofeedback is based on a result of the analyzing of the generated waveform. The display surface is used to provide the biofeedback to the monitored subject in the form of a display of the generated waveform and a target waveform.

In one embodiment, a method for providing biofeedback to reduce anxiety and stress includes performing non-contact monitoring of a subject to detect respiratory motion of a subject. The method programmatically generates a waveform based on the detected respiratory motion. The generated waveform is programmatically analyzed based upon pre-determined criteria identifying a target waveform for the subject. The method also provides biofeedback to the subject to assist the subject in obtaining or maintaining the target waveform. The biofeedback is based on a result of the analysis of the generated waveform.

### BRIEF DESCRIPTION OF THE DRAWINGS

The accompanying drawings, which are incorporated in and constitute a part of this specification, illustrate one or more embodiments of the invention and, together with the description, explain the invention. In the drawings:

FIG. 1 depicts an exemplary environment suitable for practicing embodiments of the present invention;

FIG. 2 depicts an exemplary integrated biofeedback apparatus;

FIG. 3 depicts an exemplary sequence of steps performed by an embodiment of the present invention to provide biofeedback to a monitored subject; and

FIG. 4 depicts an exemplary sequence of steps performed by an embodiment of the present invention to determine whether an intermediate waveform should be provided as biofeedback to a monitored subject.

### DETAILED DESCRIPTION

Research has demonstrated that consciously altering respiration can lead to a change in heart rate (typically a decrease) resulting in a positive impact on physiologic function, and may decrease resting catecholamine levels. Previous attempts at controlling physiologic processes have attempted to have a subject replicate an idealized breathing waveform in an effort to achieve desirable changes in heart rate and catecholamine levels. For example, some forms of meditation encourage their practitioners to practice specific breathing patterns so as to achieve a resting or meditative state consistent with an idealized waveform. Additionally, a number of previous techniques have attempted to control physiologic functions in a test subject by providing biofeedback information (information about physiological processes) to the test subject regarding cardiac function so as to achieve health benefits. One example of this approach is having the participant contact a pad measuring heart rate. The subject then tries to alter his or her breathing so that a change in heart rate is observed. This system requires direct contact to a sensor and only indirectly sees the effects of a change in respiration, which is the primary function consciously being altered.

A major drawback to previous conventional techniques providing biofeedback is that these conventional techniques require contact monitoring which create an artificial condition. The fact that the monitored subject is aware of the monitoring is counter-productive in that it tends to increase stress levels. Additionally, the use of contact monitoring also makes it difficult to monitor subjects during the course of regular (leisure and occupational) activities.

In contrast to these previous techniques, the embodiments of the present invention utilize a non-contact monitoring system to monitor physiologic functions of a monitored subject which are analyzed to provide biofeedback in real-time that is directed to reducing stress and anxiety for the monitored subject. Non-contact measurement of breathing parameters (e.g.: rate, rhythm amplitude, pauses, inspiratory to expiratory ratio), and/or cardiac parameters (e.g.: rate, rhythm, amplitude) and/or body movements are utilized to allow a subject to track, visualize and respond to these physiologic signals, in an effort to achieve an altered physiologic state that should positively impact morbidity and mortality. For example, it has been demonstrated that by consciously altering one's breathing, heart rate can be reduced, the reduction of which is believed to lead to a more favorable physiologic condition, in which catecholamine levels and oxygen consumption are reduced.

The biofeedback may include breathing, cardiac and body movement waveforms and numbers which can be viewed and analyzed by a monitored subject in real-time so that the subject can alter his or her breathing in an effort to positively impact well-being. The biofeedback may also be accompanied by audible and aromatic feedback to create a relaxing environment for the monitored subject. The biofeedback may be used by the subject to achieve a target respiratory or cardiac waveform to facilitate reductions in stress and anxiety.

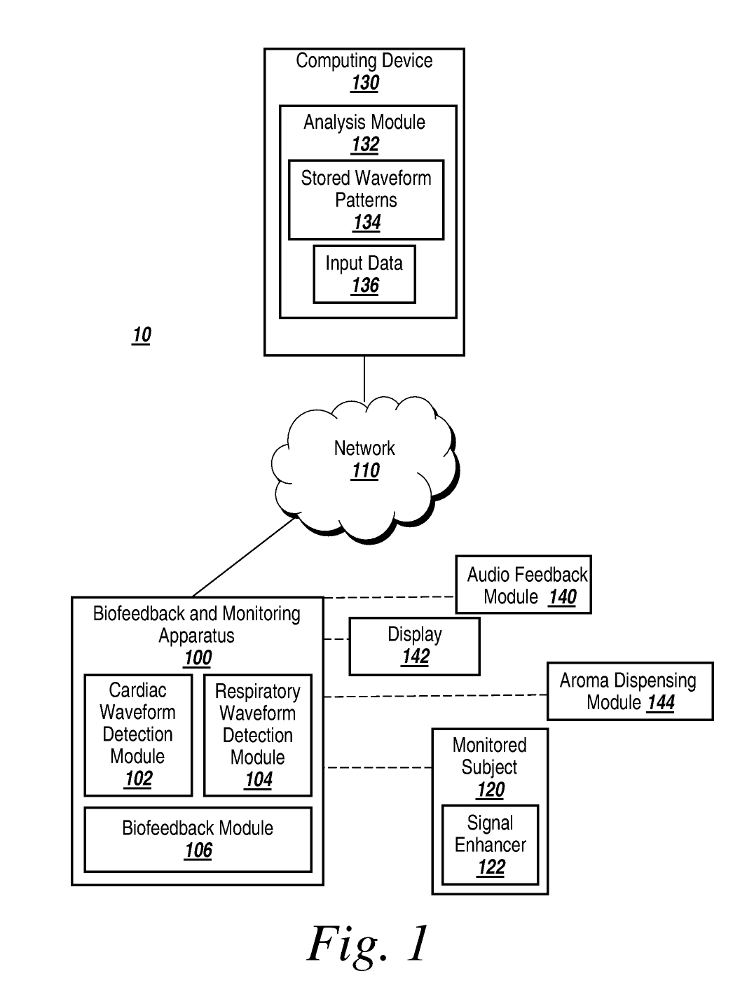

FIG. 1 depicts an exemplary environment suitable for practicing embodiments of the present invention. Biofeedback and monitoring system 10 may include biofeedback and monitoring apparatus 100 that is used to monitor physiological factors for a monitored subject 120. Biofeedback and monitoring apparatus 100 may include a respiratory waveform detection module 102. Respiratory waveform detection module 102 is used to perform non-contact respiratory monitoring of monitored subject 102 and to generate a waveform representing the monitored respiratory process. A number of different techniques to perform the non-contact monitoring may be used and are described in greater detail below.

Biofeedback and monitoring apparatus 100 may also include a cardiac waveform detection module 104. Cardiac waveform detection module 104 is used to perform non-contact cardiac monitoring of monitored subject 102 and to generate a waveform representing the monitored cardiac process. A number of different techniques to perform the non-contact cardiac monitoring may be used and are described in greater detail below.

Once a waveform representing the monitored respiratory or cardiac function has been generated, biofeedback and monitoring system 10 analyzes the generated waveform to determine whether the current monitored physiologic process is optimal. In one embodiment, the generated waveform is programmatically analyzed by a software analysis module 132 executing on a computing device 130. Computing device 130 may take many forms, including but not limited to a personal computer, workstation, server, network computer, quantum computer, optical computer, bio computer, Internet appliance, mobile device, a pager, a tablet computer, or other form of digital computer specifically configured to execute analysis module 132. Computing device 130 may be electronic and may include a Central Processing Unit (CPU), memory, storage, input control, modem, network interface, etc. The CPU may control each component of computing device 130 to provide an environment suitable for executing analysis module 132. The memory on computing device 130 temporarily stores instructions and data and provides them to the CPU so that the CPU operates the computing device 130.

Optionally, computing device 130 may include multiple CPUs for executing software loaded in memory and other programs for controlling system hardware. Each of the CPUs can be a single or a multiple core processor. The code loaded in the memory may run in a virtualized environment, such as in a Virtual Machine (VM). Multiple VMs may be resident on a single processor. Also, part of the code could be run in hardware, for example, by configuring a field programmable gate array (FPGA), using an application specific instruction set processor (ASIP) or creating an application specific integrated circuit (ASIC).

Input control for the computing device 130 may interface with a keyboard, mouse, microphone, camera, such as a web camera, or other input devices such as a 3D mouse, space mouse, multipoint touchpad, accelerometer-based device, gyroscope-based device, etc. Computing device 130 may receive, through the input control, input data relevant for calculating target waveforms for monitored subject 120. Optionally, computing device 130 may display data relevant to the generated waveform on a display as part of the analysis process.

In one embodiment, biofeedback and monitoring apparatus 100 communicates with computing device 130 over a network 110. Network 110 may be the Internet, intranet, LAN (Local Area Network), WAN (Wide Area Network), MAN (Metropolitan Area Network), wireless network or some other type of network over which biofeedback and monitoring apparatus 100 and computing device 130 can communicate. Although depicted as a separate device in FIG. 1, it should also be appreciated that computing device 130 may be part of an integrated apparatus with biofeedback and monitoring apparatus 100.

Analysis module 132 analyzes the generated waveform produced by biofeedback and monitoring apparatus 100 using pre-determined criteria. The generated waveform is compared against stored waveform patterns 134 to determine whether the current generated waveform represents an optimal waveform for the monitored physiologic process. The selection of the comparison waveform from the stored waveform patterns may utilize previous input data 136 that includes information regarding the monitored subject such as personal medical information (e.g. sex, height, weight, age, family history of various diseases, etc. and occupational information). Based on available data, the analysis module 132 selects either a customized target waveform or a default waveform for comparison to the generated waveform.

As noted, in one embodiment, the analysis of the generated waveform may be a programmatic process that occurs in an automated fashion. In an alternate embodiment, the process may also involve human input in reviewing the selection of the target waveform prior to completion of the analysis. In one embodiment, all of the analysis decisions are saved for future study in order to continually refine the stored waveform patterns 134.

The results of the analysis performed by the analysis module 132 are provided to biofeedback module 106. It should be appreciated that in some embodiments, the functionality attributed to the analysis module 132 and the biofeedback module 106 may be combined into a single module or split into additional modules without departing from the scope of the present invention. Depending upon the results of the analysis, biofeedback module 106 may take a number of actions. If the monitored subject is already exhibiting a waveform for the monitored physiologic process consistent with the desired target waveform, biofeedback module 106 may provide limited or no biofeedback. Instead, the biofeedback module 106 may provide alternative sensory feedback designed to create an environment conducive to maintaining the current respiratory or cardiac function. For example, biofeedback module 106 may provide only audible feedback such as music via audio module 140 or aromatic feedback via aroma dispensing module 144 designed to maintain the status quo. Alternatively, in such a situation, biofeedback module 106 may provide no feedback at all as the monitored subject has already achieved a desired waveform.

If the results of the analysis performed by analysis module 132 show a discrepancy between the generated waveform (representing the monitored physiologic process) and the target waveform selected by the analysis process, the biofeedback module may provide biofeedback such as providing biofeedback via a display 142 visible to monitored subject 120. The biofeedback may include graphical representations of the generated waveform and target waveform and numerical values representing current physiological measurements. In one embodiment, the two waveforms may be superimposed over each other. Biofeedback module 106 may also provide numerical data such as current respiratory and/or cardiac rates and visual instructions to monitored subject 120 suggesting monitored subject take a particular action (e.g.: begin a slow breathing exercise to attempt to control respiration rate) that will move the subject towards the target waveform.

In one embodiment, the analysis module 132 may report a significant discrepancy between the generated waveform of the monitored physiological process and the target waveform that exceeds a pre-determined parameter. In such a circumstance, biofeedback module 106 may provide an intermediate waveform to monitored subject 120 rather than the target waveform in an attempt to incrementally adjust the monitored physiological process. The intermediate waveform in such a situation may represent a more attainable goal to monitored subject 120 and its use may prevent the monitored subject from becoming alarmed (which is counter-productive) over the size of the difference between the generated and target waveforms. Biofeedback module 106 may provide a number of intermediate waveforms as appropriate for the monitored subject to attempt to replicate as part of the biofeedback in order to incrementally move the monitored subject towards his or her target waveform. The embodiments of the present invention thus provide the ability to adjust real-time non-contact biofeedback based on the subject's actual response to the intervention. This method is consistent with the movement to personalized medicine where interventions are made specific to a user, not just a population.

The non-contact monitoring system may use radiated energy (e.g.: ultrasonic, radio frequency, infrared, laser, etc.) to identify cardiac and respiratory waveforms in patients. The monitoring system may illuminate a subject in radiated energy and then detect the reflected radiated energy caused by respiratory and/or cardiac functions. The detected reflections are used to plot a two-dimensional waveform. The waveforms represent the rise and fall of a detected signal (the reflected energy) over time and are indicative of the small movements of the patient's chest, abdomen and/or other anatomical sites that are associated with respiratory and/or cardiac function. Different implementations of the monitoring system use different forms of radiated energy (e.g.: laser or ultrasonic energy) to capture breathing and cardiac waveforms for analysis. Following analysis, appropriate biofeedback is provided to the monitored subject.

One example of a suitable non-contact monitoring system that may be leveraged in conjunction with the embodiments of the present invention is described in U.S. Pat. No. 6,062,216 ('216 patent). As described in the '216 patent, a respiratory monitor may employ either ultrasonic or laser monitoring of an individual's breathing or cardiac function by measuring changes in body position with respect to time. The device continuously and without the need for contact, monitors the individual's breathing and cardiac function (and analyzes the measured waveform and identifies respiratory rate, apneic pauses, and obstructive breathing) and heart rate and rhythm, and body movements. The '216 patent (the contents of which are hereby incorporated by reference) describes a monitoring system using laser energy or ultrasonic energy to monitor respiratory function so as to detect sleep apnea but may be adapted to perform the respiratory and cardiac monitoring described herein. It should be appreciated that although the monitoring system of the '216 patent has been cited as an exemplary monitoring system which may be used in the present invention, other non-invasive monitoring systems utilizing laser or ultrasonic energy to detect respiratory and/or cardiac waveforms may also be used within the scope of the present invention.

In one embodiment, the respiratory waveform detection module 102 and the cardiac waveform detection module 104 may use ultrasound to perform the physiological monitoring may use ultrasound to establish the waveforms used in the present invention. Ultrasonic sound is a vibration at a frequency above the range of human hearing, in other words usually in a range above 20 kHz. A shaped transducer in the monitoring system radiates a preferably continuous beam of ultrasound for example in the 25 kHz to 500 kHz range to illuminate a subject patient. A receiving transducer in the monitoring system of the present invention or transducer array develops one or more signals, which shift slightly from the incident frequency due to cardiac or respiratory motion. The signal is then analyzed and plotted to generate a waveform, which may be compared against an appropriate benchmark. Appropriate adjustments are made by the monitoring system to account for the distance between the monitoring system and the subject as well as any environmental factors affecting the detection of the reflected energy.

In another embodiment, the monitoring system may use laser detection means as described in the '216 patent in place of ultrasonic energy. In such a case a laser illuminates the subject patient in a beam of light of a selected wavelength and the reflected energy, which varies, based on respiratory and/or cardiac movements is traced so as to generate a waveform. Infrared, radio frequency or other wavelengths that are highly distinct from the spectral range of other light sources surrounding the subject may be selected so as to ease the detection of the reflected energy.

In one embodiment the biofeedback module 106 displays the breathing waveform derived from the non-contact measurement of breathing. Of note, the breathing waveform can be captured through clothes and does not need a specific window to receive the necessary information to generate a breathing or cardiac waveform. However, in one embodiment, a signal enhancer 122 may be utilized to augment the reflected signal. This may be in the form of a “relaxation patch” worn by the participant.

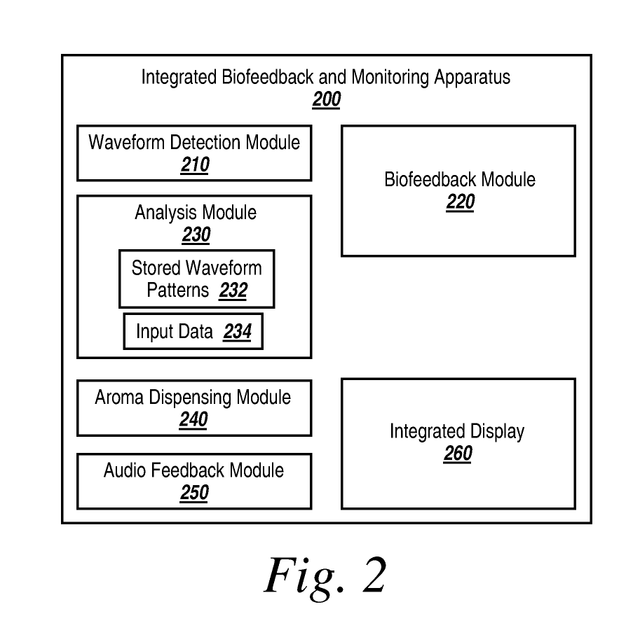

In one embodiment, the biofeedback and monitoring system described herein may be provided as an integrated biofeedback and monitoring apparatus rather than as separate components in multiple devices. FIG. 2 depicts an exemplary integrated biofeedback apparatus 200 that includes most or all of the components of the biofeedback and monitoring system described in FIG. 1. The integrated biofeedback apparatus 200 may include one or more waveform detection modules 210 such as respiratory waveform detection modules and cardiac waveform detection modules. The integrated biofeedback apparatus 200 may also include biofeedback module 220 and analysis module 230. It will be appreciated that biofeedback module 220 and analysis module 230 may be combined into a single module or split into additional modules without departing from the scope of the present invention.

Analysis module 230 may include stored waveform patterns 232 and stored input data 234 specific to a monitored subject. In one embodiment, integrated biofeedback apparatus 200 may also include an aroma dispensing module 240 an audio module 250 for providing aromatic and audio feedback and an integrated display module 260 utilized to provide biofeedback to a monitored subject in the manner described herein. In other embodiments, integrated biofeedback apparatus 200 may contain some but not all of the modules 240, 250 and 260 used to provide feedback and biofeedback. The aroma dispensing module 240 may include one or more stored scents that are designed to be soothing when inhaled and that are released into the monitored subject's environment upon a signal received from the biofeedback module 206.

In one exemplary embodiment, the integrated biofeedback apparatus 200 may be provided via a portable device such as a mobile phone or laptop. For example, the mobile phone or laptop may be equipped with an ultrasound probe that is part of the device or connected via USB that is used to perform ultrasound monitoring. Similarly, the biofeedback module and analysis modules described herein may be preinstalled or downloaded to the device. The phone or laptop display and speakers may be used to provide visual biofeedback and audio feedback respectively.

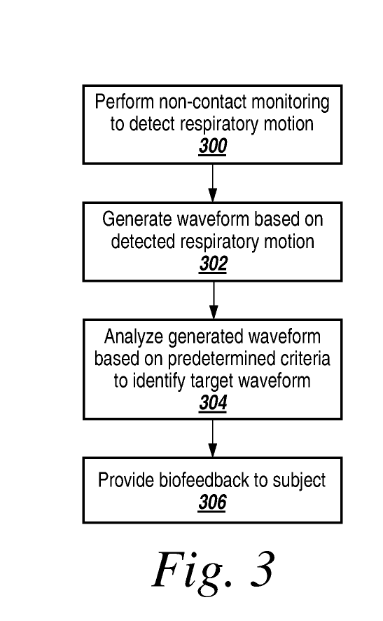

FIG. 3 depicts an exemplary sequence of steps performed by an embodiment of the present invention to provide biofeedback to a monitored subject. The sequence may begin by providing non-contact monitoring as described herein of a subject to detect respiratory motion (step 300). Of note, the subject may or may not be aware of the monitoring. In one embodiment, the subject is informed of the beginning of the monitoring and attempts to perform breathing exercises to enter a relaxed state. In another embodiment, background monitoring may be conducted as part of a normal background process. For example, the monitoring could be performed continually at work and the subject only notified and provided with biofeedback in the event that sub-optimal physiological factors were detected. The embodiments of the present invention thus provide for baseline respiratory and other parameters when affirmative relaxation exercises are not being conducted. This represents a departure from conventional biofeedback techniques that rely on intervening and changing physiologic parameters based on monitoring when the subject is conscious and focused on the monitoring. The embodiments of the present invention may thus be beneficial to background stress and anxiety reduction since long-term alterations in breathing, heart rate and the like are important for morbidity and mortality reduction. For example, during the period when relaxation exercises are not being actively being employed, should physiologic parameters be found to be out of range, soothing music, aromatic or visual therapy can be automatically instituted. The time interval at which the monitoring is conducted can be user set, set by a healthcare professional, or set based on monitored responses from the subject.

A waveform is generated as a result of the monitoring process (step 302) and analyzed based on pre-determined criteria to identify a target waveform for the monitored subject (step 304). Appropriate biofeedback is then provided to the monitored subject based on the analysis (step 306). For example, the subject's breathing, and or heart rate and body movement waveforms or numerical data may be displayed. The subject can then alter his or her breathing to idealized target patterns, which can be superimposed and displayed on the screen with the subject's actual waveforms.

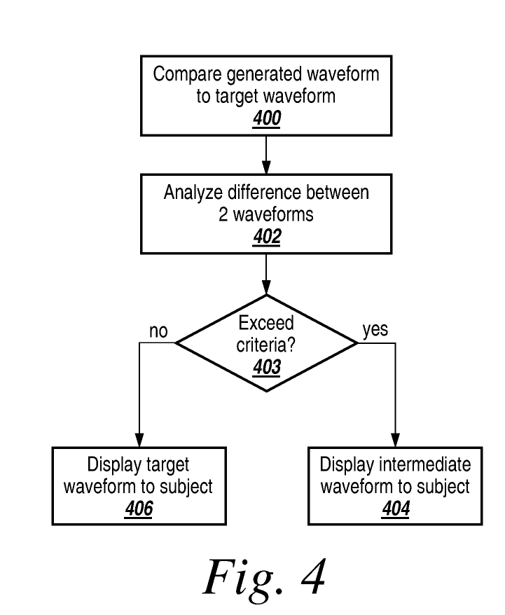

As noted above, in one embodiment the biofeedback module will determine that the monitored subject should be presented with an intermediate waveform. FIG. 4 depicts an exemplary sequence of steps performed by an embodiment of the present invention to determine whether an intermediate waveform should be provided as biofeedback to a monitored subject. The sequence begins when the generated waveform from the monitoring of the subject is compared to a target waveform (step 400). The differences between the two waveforms are analyzed (step 402) and a determination is reached as to whether or not the difference between the two waveforms exceeds pre-determined criteria (step 403). If the difference does exceed the criteria, one or more intermediate waveforms between the generated waveform and the target waveform are presented (in sequence if necessary) to the subject as part of the biofeedback (step 404). If the difference does not exceed the criteria, the target waveform is presented to the subject as usual (step 406).

Given the ability to capture and display physiologic waveforms without any direct contact to the patient, the system is well suited to apply known therapies for relaxation and stress reduction. That is, getting the participant to reduce his or her breathing rate can be coupled with visual, aromatic or auditory cues to further enhance this beneficial effect. Music or visual images can be displayed in response to both positive and negative physiologic responses on the system.

As noted, the monitored information and analysis decisions may be stored. The ability to store the monitored information allows an objective response to therapy, provides storage for medical records and is of possible importance for their party reimbursement. Further, having objective and permanent records of responses to therapy may add to the attractiveness of the technique and lead to better compliance with a relaxation regime.

It should be understood that other physiologic parameters, (video, audio, etc) could be incorporated to add robustness to the proposed system. Further, though breathing, cardiac and body movement are optimally derived through non-contact means to prevent the creation of an overly artificial environment, a contact monitoring system may also be used to perform monitoring of a subject.

The present invention may be provided as one or more computer-readable programs embodied on or in one or more physical mediums. The mediums may be a floppy disk, a hard disk, a compact disc, a digital versatile disc, a flash memory card, a PROM, an MRAM, a RAM, a ROM, or a magnetic tape. In general, the computer-readable programs may be implemented in any programming language. Some examples of languages that can be used include C, C++, C#, Python, FLASH, JavaScript, or Java. The software programs may be stored on, or in, one or more mediums as object code. Hardware acceleration may be used and all or a portion of the code may run on a FPGA, an Application Specific Integrated Processor (ASIP), or an Application Specific Integrated Circuit (ASIC). The code may run in a virtualized environment such as in a virtual machine. Multiple virtual machines running the code may be resident on a single processor.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention.

The foregoing description of example embodiments of the invention provides illustration and description, but is not intended to be exhaustive or to limit the invention to the precise form disclosed. Modifications and variations are possible in light of the above teachings or may be acquired from practice of the invention. For example, while a series of acts has been described with regard to FIGS. 3-4, the order of the acts may be modified in other implementations consistent with the principles of the invention. Further, non-dependent acts may be performed in parallel.

In addition, implementations consistent with principles of the invention can be implemented using devices and configurations other than those illustrated in the figures and described in the specification without departing from the spirit of the invention. Devices and/or components may be added and/or removed from the implementations of FIGS. 1-2 depending on specific deployments and/or applications.

The scope of the invention is defined by the claims and their equivalents.

We claim:

1. A biofeedback system for reducing anxiety and stress, comprising:
a respiratory waveform detection module, the respiratory waveform detection module performing non-contact monitoring of a subject to detect respiratory motion and generating a waveform based on the detected respiratory motion;
an analysis module programmatically analyzing the generated waveform based upon pre-determined criteria identifying a target waveform for the subject; and
a biofeedback module providing biofeedback to the subject to assist the subject in obtaining or maintaining the target waveform, the biofeedback based on a result of the analyzing of the generated waveform.

2. The system of claim 1, further comprising:
a display, the display used to provide the biofeedback in the form of a display of the generated waveform and a target waveform.

3. The system of claim 2 wherein the display of the generated waveform is accompanied by instructions for the subject to decrease a rate of breathing so as to attain the target waveform.

4. The system of claim 1, further comprising:
a display, the display used to provide the biofeedback in the form of a display of an intermediate waveform that represents a waveform between the generated waveform and the target waveform.

5. The system of claim 1 wherein the biofeedback module provides the intermediate waveform based on the analyzing indicating a difference between the target waveform and the generated waveform that is greater than pre-determined criteria.

6. The system of claim 1, further comprising:
an aroma dispensing module that is used to provide feedback in the form of aromas detectable by the subject.

7. The system of claim 1, further comprising:
an auditory module that is used to provide feedback in the form of audio transmissions detectable by the subject.

8. The system of claim 1, further comprising:
a signal enhancer, the signal enhancer affixed to the clothing of the subject and used by the respiratory waveform detection module to increase detection of respiratory motion.

9. The system of claim 1 wherein the analysis module uses personalized data concerning an occupation or physical condition of the subject in determining the target waveform.

10. The system of claim 1 wherein the respiratory waveform detection module and the analysis module communicate over a network.

11. An integrated biofeedback apparatus for reducing anxiety and stress, comprising:
a respiratory waveform detection module, the respiratory waveform detection module performing non-contact monitoring of a subject to detect respiratory motion and generating a waveform based on the detected respiratory motion;
an analysis module programmatically analyzing the generated waveform based upon pre-determined criteria identifying a target waveform for the subject;
a biofeedback module providing biofeedback to the subject to assist the subject in obtaining or maintaining the target waveform, the biofeedback based on a result of the analyzing of the generated waveform; and
a display surface, the display surface used to provide the biofeedback in the form of a display of the generated waveform and a target waveform.

12. The apparatus of claim 11, further comprising:
an auditory module that is used to provide feedback in the form of audio transmissions detectable by the subject.

13. The apparatus of claim 11, further comprising:
an aroma dispensing module that is used to provide feedback in the form of aromas detectable by the subject.

14. The apparatus of claim 11 wherein the respiratory waveform detection module monitors the subject using radiated energy.

15. The apparatus of claim 14 wherein the respiratory waveform detection module monitors the subject using ultrasound.

16. The apparatus of claim 14 wherein the respiratory waveform detection module monitors the subject using laser detection means.

17. The apparatus of claim 14 wherein the respiratory waveform detection module monitors the subject using infrared or radio frequency transmissions.

18. A method for providing biofeedback to reduce anxiety and stress, comprising:
performing non-contact monitoring of a subject to detect respiratory motion of a subject;
generating programmatically a waveform based on the detected respiratory motion;
analyzing programmatically the generated waveform based upon pre-determined criteria identifying a target waveform for the subject; and
providing biofeedback to the subject to assist the subject in obtaining or maintaining the target waveform, the biofeedback based on a result of the analyzing of the generated waveform.

19. The method of claim 18, further comprising:
providing the biofeedback in the form of a display of the generated waveform and a target waveform.

20. The method of claim 19, further comprising:
conveying instructions for the subject to decrease a rate of breathing so as to attain the target waveform.

21. The method of claim 18, further comprising:
providing the biofeedback in the form of a display of an intermediate waveform that represents a waveform between the generated waveform and the target waveform.

22. The method of claim 21 wherein the intermediate waveform is displayed based on the analyzing indicating a difference between the target waveform and the generated waveform that is greater than pre-determined criteria.

23. The method of claim 18, further comprising:
providing feedback in the form of an aroma detectable by the subject.

24. The method of claim 18, further comprising:
providing feedback in the form of an audio transmission detectable by the subject.

25. The method of claim 18 wherein the analyzing uses personalized data concerning an occupation or physical condition of the subject in determining the target waveform.

26. The method of claim 18 wherein the biofeedback is provided prior to the subject becoming aware of the monitoring.

27. A physical computer-readable medium holding computer-executable instructions for providing biofeedback to reduce anxiety and stress, the instructions when executed causing one or more devices to:
perform non-contact monitoring of a subject to detect respiratory motion of a subject;
generate programmatically a waveform based on the detected respiratory motion;
analyze programmatically the generated waveform based upon pre-determined criteria identifying a target waveform for the subject; and
provide biofeedback to the subject to assist the subject in obtaining or maintaining the target waveform, the biofeedback based on a result of the analyzing of the generated waveform.

28. The medium of claim 27 wherein the execution of the instructions further causes the one or more devices to:
provide the biofeedback in the form of a display of the generated waveform and a target waveform.

29. The medium of claim 28 wherein the execution of the instructions further causes the one or more devices to:
convey instructions for the subject to decrease a rate of breathing so as to attain the target waveform.

30. The medium of claim 27 wherein the execution of the instructions further causes the one or more devices to:
provide the biofeedback in the form of a display of an intermediate waveform that represents a waveform between the generated waveform and the target waveform.

31. The medium of claim 27 wherein the execution of the instructions further causes the one or more devices to:
provide feedback in the form of an aroma detectable by the subject.

32. The medium of claim 27 wherein the execution of the instructions further causes the one or more devices to:
provide feedback in the form of an audio transmission detectable by the subject.

33. The medium of claim 27 wherein the analyzing uses personalized data concerning an occupation or physical condition of the subject in determining the target waveform.
